(19) United States
(12) Patent Application Publication
(10) Pub. No.: US 2026/0194510 A1
(43) Pub. Date: Jul. 9, 2026

(54) PORTABLE TESTING SYSTEM FOR RAPID FUEL PURITY

(71) Applicant: GM Global Technology Operations LLC, Detroit, MI (US)

(72) Inventors: Jie Kou, Novi, MI (US); Steven Falta, Honeoye Falls, NY (US)

(73) Assignee: GM Global Technology Operations LLC, Detroit, MI (US)

(21) Appl. No.: 19/014,059

(22) Filed: Jan. 8, 2025

(51) Int. Cl. G01N 33/22; G01N 1/22; G01N 33/00

(52) U.S. Cl. CPC G01N 33/225; G01N 1/2247; G01N 33/0016

(57) ABSTRACT

A portable purity testing system for fuel used in a vehicle comprising a fluid conduit extending between a first end and a second end, a pressure and flow regulation system communicatively coupled to and arranged downstream of the first end, and a gas analyzer communicatively coupled to and arranged downstream of the pressure and flow regulation system.

### INTRODUCTION

The information provided in this section is for the purpose of generally presenting the context of the disclosure. Work of the presently named inventors, to the extent it is described in this section, as well as aspects of the description that may not otherwise qualify as prior art at the time of filing, are neither expressly nor impliedly admitted as prior art against the present disclosure.

The present disclosure relates generally to a portable testing system for fuel used in vehicles.

Proton exchange membrane (PEM) fuel cells are a type of fuel cell that convert chemical energy from hydrogen into electrical energy through an electrochemical reaction. These fuel cells are highly efficient and can be used in various applications, including transportation and stationary power generation.

Current fuel testing methods are typically confined to laboratory settings, which can be both time-consuming and impractical for real-time monitoring in operational environments. Shortcomings of existing systems and methods will be addressed by one or more aspects of the present disclosure.

### SUMMARY

In one configuration, a portable purity testing system for fuel used in a vehicle is provided and includes a fluid conduit extending between a first end and a second end, a pressure and flow regulation system communicatively coupled to and arranged downstream of the first end, and a gas analyzer communicatively coupled to and arranged downstream of the pressure and flow regulation system.

The portable purity testing system may include one or more of the following optional aspects or steps. For example, a first valve can be communicatively coupled to the fluid conduit at the first end. The gas analyzer can be calibrated with a calibration cylinder communicatively coupled to the first valve.

According to at least one example, a second valve can be communicatively coupled to the fluid conduit at the second end. The first valve and the second valve can both be three-way valves and the second valve can be communicatively coupled to a vent.

According to at least one aspect, the fluid conduit can be communicatively grounded at the first end.

According to another aspect, the pressure and flow regulation system can include a pressure regulator, a pressure relief valve, and a pressure gauge. The pressure and flow regulation system can further include a needle valve.

According to at least one example, the portable purity testing system further includes a sample cylinder selectively coupled to the fluid conduit downstream of the gas analyzer and having an inlet valve and an outlet valve.

According to another example, the gas analyzer includes a data interface powered by a low-voltage power source.

In one configuration, a method of auditing a fuel source with a portable purity testing system is provided and includes connecting a fluid conduit to a fuel source grounding point, setting a first valve to a desired inlet position, setting a second valve to a desired outlet position, connecting the portable purity testing system to a power source, connecting a fuel source to the portable purity testing system, adjusting a pressure and flow regulation system communicatively coupled to a gas analyzer of the portable purity testing system, supplying power to the portable purity testing system, initiating a measurement of the fuel source, purging the portable purity testing system until the gas analyzer reaches a steady state condition, and determining whether the fuel meets a minimum purity requirement.

The method may include one or more of the following optional aspects or steps. For example, setting the first valve to the desired inlet position can further include adjusting a first three-way valve to receive fuel from one of a fuel station and a fuel carrier. Setting the second valve to the desired outlet position can further include adjusting a second three-way valve to a vent flow path.

According to one aspect, adjusting the pressure and flow regulation system can further include adjusting a pressure regulator and a needle valve.

According to another aspect, determining whether the fuel meets the minimum purity requirement can further include evaluating a data interface communicatively coupled to the gas analyzer.

In another configuration, a method of collecting a sample of fuel with a portable purity testing system is provided and includes connecting a fluid conduit to a fuel source grounding point, setting a first valve to a desired inlet position, setting a second valve to a desired outlet position, connecting the portable purity testing system to a power source, connecting a fuel source to the portable purity testing system, connecting a sample cylinder to the second valve, adjusting a pressure and flow regulation system communicatively coupled to a gas analyzer of the portable purity testing system, supplying power to the portable purity testing system, initiating a measurement of the fuel source, purging the portable purity testing system until the gas analyzer reaches a steady state condition, and removing the sample cylinder from the second valve.

The method may include one or more of the following optional aspects or steps. For example, setting the first valve to the desired inlet position can further include adjusting a first three-way valve to receive fuel from one of a fuel station and a fuel carrier. Setting the second valve to the desired outlet position can further include adjusting a second three-way valve to a sample collection path.

According to at least one aspect, connecting the sample cylinder to the second valve further includes opening an inlet valve and an outlet valve of the sample cylinder. Removing the sample cylinder from the second valve can further include closing inlet and outlet valves of the sample cylinder.

### BRIEF DESCRIPTION OF THE DRAWINGS

The drawings described herein are for illustrative purposes only of selected configurations and are not intended to limit the scope of the present disclosure.

FIG. 1 is a side view of a vehicle according to principles of the present disclosure;

FIG. 2 is a schematic view of a portable testing system according to the principles of the present disclosure;

FIG. 3 is a flow diagram of a method of using the portable testing system of FIG. 2 according to principles of the present disclosure; and

FIG. 4 is a flow diagram of a method of using the portable testing system of FIG. 2 according to principles of the present disclosure.

Corresponding reference numerals indicate corresponding parts throughout the drawings.

### DETAILED DESCRIPTION

Example configurations will now be described more fully with reference to the accompanying drawings. Example configurations are provided so that this disclosure will be thorough, and will fully convey the scope of the disclosure to those of ordinary skill in the art. Specific details are set forth such as examples of specific components, devices, and methods, to provide a thorough understanding of configurations of the present disclosure. It will be apparent to those of ordinary skill in the art that specific details need not be employed, that example configurations may be embodied in many different forms, and that the specific details and the example configurations should not be construed to limit the scope of the disclosure.

The terminology used herein is for the purpose of describing particular exemplary configurations only and is not intended to be limiting. As used herein, the singular articles “a,” “an,” and “the” may be intended to include the plural forms as well, unless the context clearly indicates otherwise. The terms “comprises,” “comprising,” “including,” and “having,” are inclusive and therefore specify the presence of features, steps, operations, elements, and/or components, but do not preclude the presence or addition of one or more other features, steps, operations, elements, components, and/or groups thereof. The method steps, processes, and operations described herein are not to be construed as necessarily requiring their performance in the particular order discussed or illustrated, unless specifically identified as an order of performance. Additional or alternative steps may be employed.

When an element or layer is referred to as being “on,” “engaged to,” “connected to,” “attached to,” or “coupled to” another element or layer, it may be directly on, engaged, connected, attached, or coupled to the other element or layer, or intervening elements or layers may be present. In contrast, when an element is referred to as being “directly on,” “directly engaged to,” “directly connected to,” “directly attached to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

The terms “first,” “second,” “third,” etc. may be used herein to describe various elements, components, regions, layers and/or sections. These elements, components, regions, layers and/or sections should not be limited by these terms. These terms may be only used to distinguish one element, component, region, layer or section from another region, layer or section. Terms such as “first,” “second,” and other numerical terms do not imply a sequence or order unless clearly indicated by the context. Thus, a first element, component, region, layer or section discussed below could be termed a second element, component, region, layer or section without departing from the teachings of the example configurations.

In this application, including the definitions below, the term “module” may be replaced with the term “circuit.” The term “module” may refer to, be part of, or include an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); a processor (shared, dedicated, or group) that executes code; memory (shared, dedicated, or group) that stores code executed by a processor; other suitable hardware components that provide the described functionality; or a combination of some or all of the above, such as in a system-on-chip.

The term “code,” as used above, may include software, firmware, and/or microcode, and may refer to programs, routines, functions, classes, and/or objects. The term “shared processor” encompasses a single processor that executes some or all code from multiple modules. The term “group processor” encompasses a processor that, in combination with additional processors, executes some or all code from one or more modules. The term “shared memory” encompasses a single memory that stores some or all code from multiple modules. The term “group memory” encompasses a memory that, in combination with additional memories, stores some or all code from one or more modules. The term “memory” may be a subset of the term “computer-readable medium.” The term “computer-readable medium” does not encompass transitory electrical and electromagnetic signals propagating through a medium, and may therefore be considered tangible and non-transitory memory. Non-limiting examples of a non-transitory memory include a tangible computer readable medium including a nonvolatile memory, magnetic storage, and optical storage.

The apparatuses and methods described in this application may be partially or fully implemented by one or more computer programs executed by one or more processors. The computer programs include processor-executable instructions that are stored on at least one non-transitory tangible computer readable medium. The computer programs may also include and/or rely on stored data.

A software application (i.e., a software resource) may refer to computer software that causes a computing device to perform a task. In some examples, a software application may be referred to as an “application,” an “app,” or a “program.” Example applications include, but are not limited to, system diagnostic applications, system management applications, system maintenance applications, word processing applications, spreadsheet applications, messaging applications, media streaming applications, social networking applications, and gaming applications.

The non-transitory memory may be physical devices used to store programs (e.g., sequences of instructions) or data (e.g., program state information) on a temporary or permanent basis for use by a computing device. The non-transitory memory may be volatile and/or non-volatile addressable semiconductor memory. Examples of non-volatile memory include, but are not limited to, flash memory and read-only memory (ROM)/programmable read-only memory (PROM)/erasable programmable read-only memory (EPROM)/electronically erasable programmable read-only memory (EEPROM) (e.g., typically used for firmware, such as boot programs). Examples of volatile memory include, but are not limited to, random access memory (RAM), dynamic random access memory (DRAM), static random access memory (SRAM), phase change memory (PCM) as well as disks or tapes.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” and “computer-readable medium” refer to any computer program product, non-transitory computer readable medium, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

Various implementations of the systems and techniques described herein can be realized in digital electronic and/or optical circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

The processes and logic flows described in this specification can be performed by one or more programmable processors, also referred to as data processing hardware, executing one or more computer programs to perform functions by operating on input data and generating output. The processes and logic flows can also be performed by special purpose logic circuitry, e.g., an FPGA (field programmable gate array) or an ASIC (application specific integrated circuit). Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, one or more aspects of the disclosure can be implemented on a computer having a display device, e.g., a CRT (cathode ray tube), LCD (liquid crystal display) monitor, or touch screen for displaying information to the user and optionally a keyboard and a pointing device, e.g., a mouse or a trackball, by which the user can provide input to the computer. Other kinds of devices can be used to provide interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

According to principles of the present disclosure, a portable purity testing system is provided and can be configured for in-situ hydrogen gas (H2) testing for fuel stations and fuel carriers, such as bottles and trailers in the field. Gas samples are ordinarily tested in a laboratory, but this procedure can be time consuming and expensive. Identifying low quality fuel at a source (e.g., a fuel station, bottle, trailer, etc.) in the field is desirable to avoid catastrophic damage to fuel cell systems. The portable purity testing system can be configured so that a sample can be obtained for further testing.

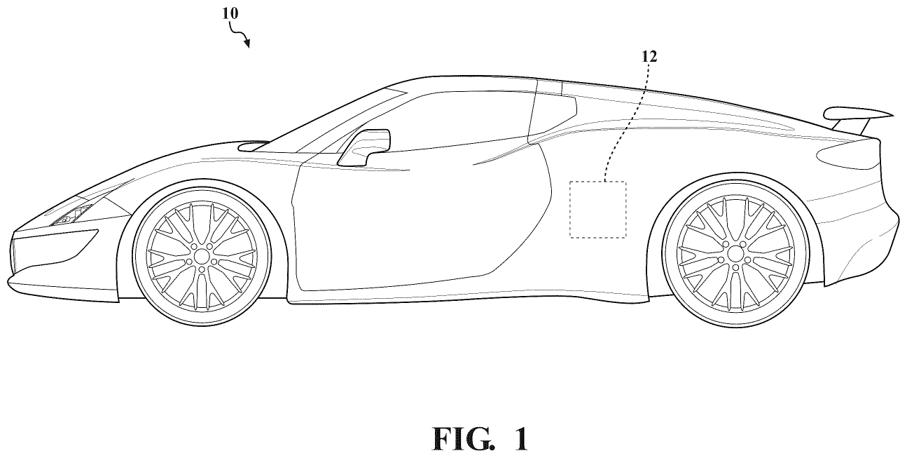

With reference to FIG. 1, an illustrative example of a vehicle 10 is provided in accordance with the principles of the present disclosure. The vehicle 10 is a hydrogen fuel cell vehicle (FCV) that uses hydrogen gas as a primary fuel source. In general, the hydrogen gas can be converted into electricity through a chemical reaction within a fuel cell stack 12 arranged in or on the vehicle 10. Other vehicles (e.g., trains, planes, boats, etc.) and applications (e.g., stationary generators, etc.) can benefit from the principles of the present disclosure as well.

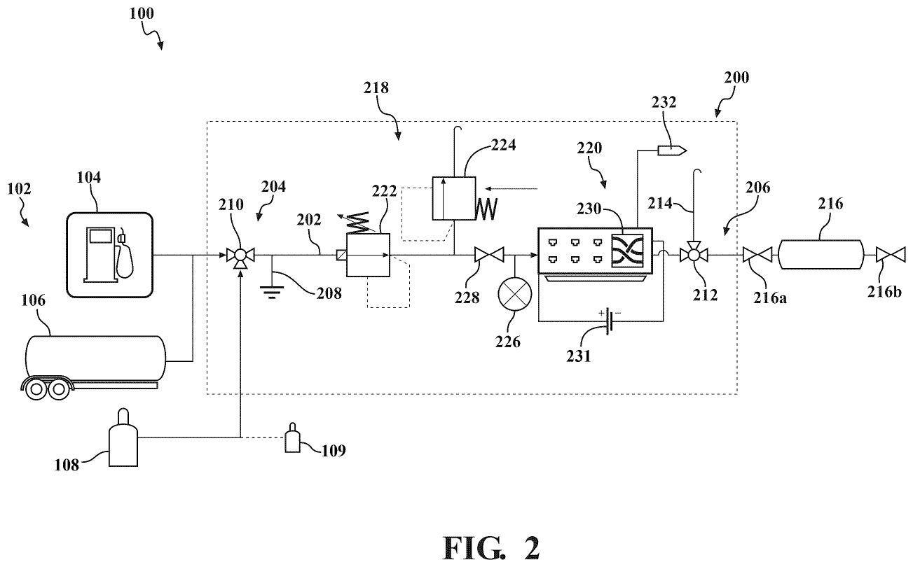

With reference to FIG. 2, an operating environment 100 is provided and includes several fuel sources 102 and a portable purity testing system 200 (hereinafter, “testing system”). The fuel sources 102 can include fuel stations 104 and fuel carriers, such as fuel trailers 106 and portable fuel tanks 108. While the present disclosure discusses the testing system 200 with respect to the fuel sources 102, other fuel sources not listed here may be used as well. For instance, fuel stored in a tank of the vehicle 10 can be tested using the testing system 200 as well.

With continued reference to FIG. 2, the testing system 200 includes a fluid conduit 202 extending between a first end 204 and a second end 206 that is arranged downstream of the first end 204. The fluid conduit 202 can be communicatively grounded at 208 to provide a pathway for any static electricity that may have built up in the testing system 200 during transport, for example. In the present illustrative example, the fluid conduit 202 is communicatively grounded near or at the first end 204 for electrostatic discharge. A first valve 210 can be communicatively coupled to the fluid conduit 202 at the first end 204 and can be configured to quickly connect and disconnect from the one or more fuel sources 102. For instance, a three-way valve can be used so that the testing system 200 can be coupled to and receive gas from the fuel stations 104, the fuel trailers 106, and/or the fuel tanks 108. Optionally, the first valve 210 can be configured to receive gas from a calibration cylinder 109 that contains a sample of gas with a known purity. A second valve 212 can be communicatively coupled to the fluid conduit 202 at the second end 206. The second valve 212 can be a three-way valve or another valve that includes a vent 214 and an attachment that can receive a sample cylinder 216. As will be discussed below, the vent 214 provides a pathway for purging any residual air and/or other gases that may be disposed in the testing system 200 from prior connections. Additionally, the vent 214 allows a continuous sample to flow from one end of the testing system 200 to the other. In one configuration, the sample cylinder 216 includes an inlet valve 216a and an outlet valve 216b. The inlet and outlet valves 216a, 216b are desirable so that the sample cylinder 216 can be purged before a sample of fuel is collected.

The testing system 200 includes a flow and pressure regulation system 218 (hereinafter, “regulation system”) that can be adjusted to establish a pressure and/or flow rate that is acceptable for a gas analyzer 220 arranged downstream of the regulation system 218. According to one aspect, the regulation system 218 can be adjusted manually by a user and/or by an automated system (not shown) communicatively coupled to the regulation system 218. With reference to FIG. 2, the regulation system 218 includes a pressure regulator 222 that is configured to maintain a constant output pressure regardless of variation of input pressure or flow rate. The pressure regulator 222 can be desirable for protecting and ensuring consistent operation of the gas analyzer 220. For example, the fuel sources 102 can have a supply pressure of up to 900 bar, so the pressure regulator 222 can be desirable to step down (i.e., reduce) the pressure to a level that is acceptable for the gas analyzer 220. Optionally, a pressure relief valve 224 can be arranged downstream of the pressure regulator 222 to release excess pressure before it reaches the gas analyzer 220. The pressure relief valve 224 can be desirable to protect the gas analyzer 220 from uncontrolled pressure if the pressure regulator 222 fails, for example. A pressure gauge 226 can be arranged downstream of the pressure regulator 222 and/or the pressure relief valve 224 so that a user can determine the pressure entering the gas analyzer 220, for example. The regulation system 218 can further include a needle valve 228 that can be adjusted manually or otherwise to establish a flow rate that is acceptable for the gas analyzer 220, for example.

The gas analyzer 220 can be configured to provide a real-time hydrogen gas purity reading (i.e., measurement, value, etc.) and/or report that indicates whether the quality of the hydrogen gas is acceptable. The gas analyzer 220 can be configured with a data interface 230 that an operator can rely on to control the gas analyzer 220 and to instantaneously assess results during and/or after testing. According to one aspect, the gas analyzer 220 can be configured to report hydrogen gas purity accuracy at +/−0.01% with accredited lab calibration traceability. To maintain this level of accuracy, as introduced above, the gas analyzer 220 can be regularly calibrated using the calibration cylinder 109. The gas analyzer 220 can be powered using a low voltage power source, such as a direct current (DC) battery power supply 231. Additionally, in at least one configuration, the gas analyzer 220 can include a power switch 232 that controls the flow of power between the battery 231 and the gas analyzer 220.

The testing system 200 can be mounted on a vehicle (e.g., a maintenance vehicle) or packaged in a case that can be easily transported throughout the operating environment 100.

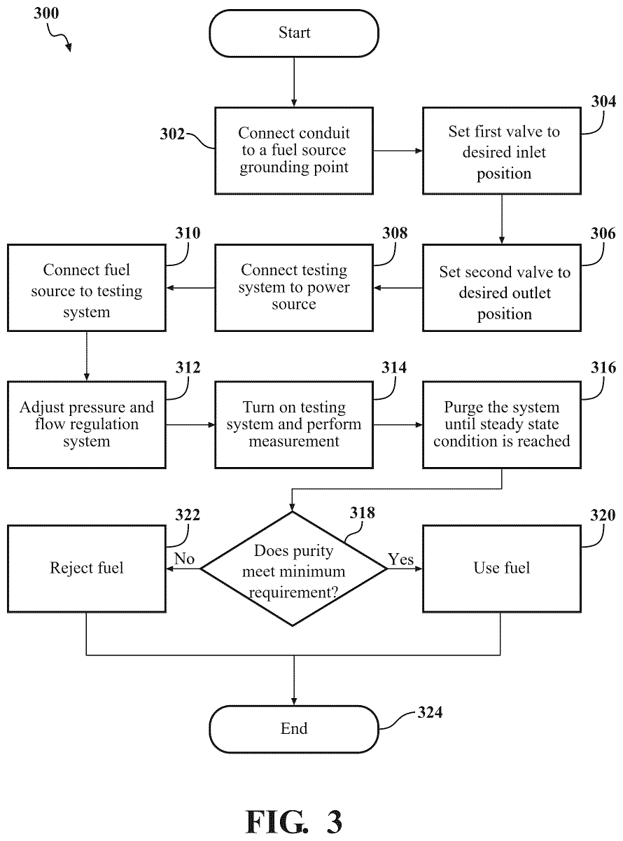

With reference to FIG. 3, a method 300 of auditing one of the fuel sources 102 in the operating environment 100 with the testing system 200 is provided.

At 302, the testing system 200 (e.g., the fluid conduit 202) is grounded to a fuel source grounding point at 208.

At 304, the first valve 210 is set to a desired inlet option. Depending on what type of fuel source 102 is being tested, a user can manipulate the first valve 210 to the desired pathway so that fuel will flow from one of the fuel station 104, the fuel trailers 106, or the fuel tanks 108 into the testing system 200 upon connection.

At 306, the second valve 212 is set to a venting path coupled to the vent 214. In doing so, the fluid conduit 202 is open between the first end 204 and the second end 206.

At 308, the power source is connected to the testing system 200. In the present illustrative example, the battery 231 is communicatively coupled to the testing system 200.

At 310, one of the fuel sources 102 is coupled to the first valve 210. In other words, one of the fuel stations 104, the fuel trailers 106, or the fuel tanks 108 is communicatively coupled to the testing system 200 and fuel begins to flow through the fluid conduit 202 from the first end 204 to the second end 206.

At 312, the regulation system can be manually or automatically maintained. In either case, the pressure regulator 222 and the needle valve 228 are adjusted to achieve a pressure and flow that is acceptable for the gas analyzer 220.

At 314, the gas analyzer 220 is powered on. Practically speaking, an operator can manipulate the power switch 232 to an “on” position.

At 316, testing (i.e., a purity measurement) is initiated and the testing system 200 is purged until the gas analyzer 220 has reached steady state.

At 318, a user or the data interface 230 can determine whether the purity of the hydrogen gas meets a minimum purity requirement.

At 320, the fuel is used if it meets the minimum purity requirement.

At 322, the fuel is rejected if it does not meet the minimum purity requirement.

At 324, the method 300 ends.

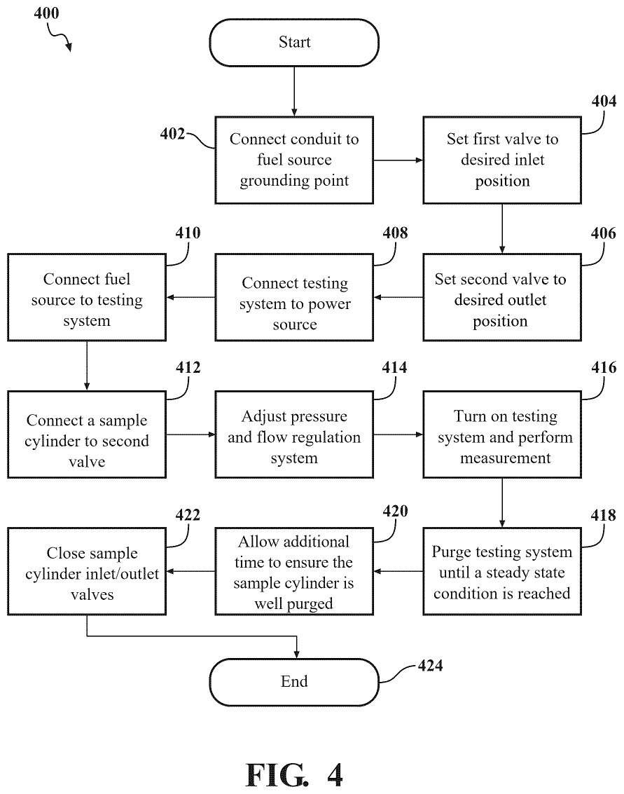

With reference to FIG. 4, a method 400 for collecting a sample from one of the fuel sources 102 in the operating environment 100 with the testing system 200 is provided.

At 402, the testing system 200 (e.g., the fluid conduit 202) is grounded to a fuel source grounding point at 208.

At 404, the first valve 210 is set to a desired inlet option. Depending on what type of fuel source 102 is being tested, a user can manipulate the first valve 210 to a desired pathway so that fuel will flow from one of the fuel stations 104, the fuel trailers 106, or the fuel tanks 108 into the testing system 200 upon connection.

At 406, the second valve 212 is set to a sample collection path which will allow fuel to flow through the second end 206 of the fluid conduit 202 and out of the second valve 212.

At 408, the sample cylinder 216 is communicatively coupled to the second valve 212 so that gas can flow from the fluid conduit 202 and into the sample cylinder 216. The inlet valves 216a and the outlet valve 216b can be opened so that fuel can enter the inlet valve 216a, travel through the sample cylinder 216, and exit through the outlet valve 216b.

At 410, the power source is connected to the testing system 200. In the present illustrative example, the battery 231 is communicatively coupled to the testing system 200.

At 412, one of the fuel sources 102 is coupled to the first valve 210. In other words, one of the fuel stations 104, the fuel trailers 106, or the fuel tanks 108 is communicatively coupled to the testing system 200 and fuel begins to flow through the fluid conduit 202 from the first end 204 and toward the second end 206.

At 414, the regulation system 218 can be manually or automatically maintained. In either case, the pressure regulator 222 and the needle valve 228 are adjusted to achieve a pressure and flow that is acceptable for the gas analyzer 220.

At 416, the gas analyzer 220 is powered on and testing (i.e., a purity measurement) is initiated. Practically speaking, an operator can manipulate the power switch 232 to an “on” position and can engage with the data interface 230 to initiate testing of the fuel passing through the testing system 200.

At 418, the testing system 200 is purged until the gas analyzer 220 reaches a steady state condition. In other words, the testing system 200 is purged until a consistent purity measurement has been reached.

At 420, fuel should continue to pass through the gas analyzer 220 and the sample cylinder 216 to ensure that the sample cylinder 216 is well purged.

At 422, the inlet valve 216 a and the outlet valve 216 b of the sample cylinder 216 are closed to capture a sample of the fuel. The sample cylinder 216 can be transported to an offsite laboratory for further testing, for example.

At 424, the method 400 ends.

A number of implementations have been described. Nevertheless, it will be understood that various modifications may be made without departing from the spirit and scope of the disclosure. Accordingly, other implementations are within the scope of the following claims.

The foregoing description has been provided for purposes of illustration and description. It is not intended to be exhaustive or to limit the disclosure. Individual elements or features of a particular configuration are generally not limited to that particular configuration, but, where applicable, are interchangeable and can be used in a selected configuration, even if not specifically shown or described. The same may also be varied in many ways. Such variations are not to be regarded as a departure from the disclosure, and all such modifications are intended to be included within the scope of the disclosure.

What is claimed is:

1. A portable purity testing system for fuel used in a vehicle, comprising:
a fluid conduit extending between a first end and a second end;
a pressure and flow regulation system communicatively coupled to and arranged downstream of the first end; and
a gas analyzer communicatively coupled to and arranged downstream of the pressure and flow regulation system.

2. The portable purity testing system of claim 1, wherein a first valve is communicatively coupled to the fluid conduit at the first end.

3. The portable purity testing system of claim 2, wherein the gas analyzer is calibrated with a calibration cylinder communicatively coupled to the first valve.

4. The portable purity testing system of claim 2, wherein a second valve is communicatively coupled to the fluid conduit at the second end.

5. The portable purity testing system of claim 3, wherein the first valve and the second valve are both three-way valves and the second valve is communicatively coupled to a vent.

6. The portable purity testing system of claim 1, wherein the fluid conduit is communicatively grounded at the first end.

7. The portable purity testing system of claim 1, wherein the pressure and flow regulation system includes a pressure regulator, a pressure relief valve, and a pressure gauge.

8. The portable purity testing system of claim 7, wherein the pressure and flow regulation system includes a needle valve.

9. The portable purity testing system of claim 1, further including a sample cylinder selectively coupled to the fluid conduit downstream of the gas analyzer and having an inlet valve and an outlet valve.

10. The portable purity testing system of claim 1, wherein the gas analyzer includes a data interface powered by a low-voltage power source.

11. A method of auditing a fuel source with a portable purity testing system, comprising:
connecting a fluid conduit to a fuel source grounding point;
setting a first valve to a desired inlet position;
setting a second valve to a desired outlet position;
connecting the portable purity testing system to a power source;
connecting a fuel source to the portable purity testing system;
adjusting a pressure and flow regulation system communicatively coupled to a gas analyzer of the portable purity testing system;
supplying power to the portable purity testing system;
initiating a measurement of the fuel source;
purging the portable purity testing system until the gas analyzer reaches a steady state condition; and
determining whether the fuel source meets a minimum purity requirement.

12. The method of claim 11, wherein setting the first valve to the desired inlet position further includes adjusting a first three-way valve to receive fuel from one of a fuel station and a fuel carrier.

13. The method of claim 12, wherein setting the second valve to the desired outlet position further includes adjusting a second three-way valve to a vent flow path.

14. The method of claim 11, wherein adjusting the pressure and flow regulation system further includes adjusting a pressure regulator and a needle valve.

15. The method of claim 11, wherein determining whether the fuel source meets the minimum purity requirement further includes evaluating a data interface communicatively coupled to the gas analyzer.

16. A method of collecting a sample of fuel with a portable purity testing system, comprising:
connecting a fluid conduit to a fuel source grounding point;
setting a first valve to a desired inlet position;
setting a second valve to a desired outlet position;
connecting the portable purity testing system to a power source;
connecting a fuel source to the portable purity testing system;
connecting a sample cylinder to the second valve;
adjusting a pressure and flow regulation system communicatively coupled to a gas analyzer of the portable purity testing system;
supplying power to the portable purity testing system;
initiating a measurement of the fuel source;
purging the portable purity testing system until the gas analyzer reaches a steady state condition; and
removing the sample cylinder from the second valve.

17. The method of claim 16, wherein setting the first valve to the desired inlet position further includes adjusting a first three-way valve to receive fuel from one of a fuel station and a fuel carrier.

18. The method of claim 17, wherein setting the second valve to the desired outlet position further includes adjusting a second three-way valve to a sample collection path.

19. The method of claim 16, wherein connecting the sample cylinder to the second valve further includes opening an inlet valve and an outlet valve of the sample cylinder.

20. The method of claim 19, wherein removing the sample cylinder from the second valve further includes closing inlet and outlet valves of the sample cylinder.
